(19) United States
(12) Patent Application Publication
(10) Pub. No.: US 2007/0218129 A1
(43) Pub. Date: Sep. 20, 2007

(54) Solid Dispersible and/or Orodispersible Non-Filmy Containing at Least One Type of Active Substance Pharmaceutical Composition and Method for the Preparation Thereof

(75) Inventor: Jerome Besse, Listrac Medoc (FR)

Correspondence Address: ARENT FOX PLLC, 1050 CONNECTICUT AVENUE, N.W., SUITE 400, WASHINGTON, DC, 20036, US

(73) Assignees: SARL GALENIX INNOVATIONS, Saint-Jean D'lIIac (FR); PHARMINNOVATION, Pessac (FR)

(21) Appl. No.: 10/567,345

(22) PCT Filed: Aug. 5, 2004
(86) PCT No.: PCT/FR04/50376

§ 371 Date: Oct. 31, 2006

(30) Foreign Application Priority Data
Aug. 6, 2003 (FR) 0350403

(51) Int. Cl. A61K 9/20

(52) U.S. Cl. 424/465

(57) ABSTRACT

The invention relates to an immediate release dispersible and orodispersible solid pharmaceutical composition having the form of particles with a size lower than 710 μm, containing the metformin active ingredient, characterized in that it comprises: a) from 65% to 90% in weight of the metformin active ingredient, optionally under the form of a salt, or a combination of the metformin active ingredient with a hypoglycemic active ingredient; b) from 0.5 to 4% in weight of a binding agent or a combination of binding agents; c) from 1% to 12% in weight of a disintegrating agent or a combination of disintegrating agents; d) from 0% to 10% in weight of a diluting agent or a combination of diluting agents; e) from 0.05% to 3% in weight of a sweetening agent or a combination of sweetening agents; and f) one or more additional excipients, the weight percentages being expressed based on the total weight of said composition.

### FIELD OF THE INVENTION

The present invention relates to the development of solid pharmaceutical compositions based on metformin active ingredient, more specifically in the form of fast disintegration dispersible or orodispersible tablets.

### STATE OF THE ART

1,1-dimethylbiguanide, referred to as Metformin according to the International Common Designation (ICD), is a compound simultaneously inducing a decrease in the glucose production and an increase in its consumption in the body. Metformin is also known for inhibiting lipolysis. Metformin is used in the therapeutic field as a normoglycemic or a hypoglycemic active ingredient. More particularly, metformin is commonly used for treating hyperglycemia, non insulin-dependent diabetes, whether associated or not with obesity, and possibly for insulin-requiring diabetes as well as insulin-dependent diabetes.

Metformin can be presented under the form of a salt. U.S. Pat. No. 3,174,921 discloses several metformin salts, such as phosphate, sulphate, hydrochloride, salicylate, maleate, benzoate, ethanedisulfonate, fumarate and glycolate salts. U.S. Pat. No. 6,031,004 discloses dibasic metformin salts where the molar ratio metformin:dibasic acid is 2:1, such as, for example, fumarate and succinate dibasic salts.

Generally, in known pharmaceutical compositions, metformin is included under the form of a metformin salt such as hydrochloride, chlorophenoxyacandate or 4,4′-methylenebis(3-hydroxy-2-naphthoate) as well, the latter salt being commonly referred to as embonate.

Metformin is an active ingredient that exerts completely its normoglycemic or hypoglycemic activity only when it is administered at unit doses higher than 500 mg, more preferably higher than 800 mg.

Various metformin-based pharmaceutical compositions under the form of tablets for oral administration have been disclosed.

Those commonly marketed compositions have the form of coated or film-coated tablets, or scored tablets, such tablets conventionally being dosed with 500 to 850 mg metformin.

One of the technical disadvantages of metformin, for preparing tablets, is the low compressibility of such active ingredient, associated with a low binding capability.

In order to overcome the above-mentioned disadvantages of mefformin, it has been suggested to produce tablets using a method including a dry granulation step or a direct compression method, as in US Patent Applications 2003/0021841 and 2003/0104049. US 2003/0021841 Application relates to time-controlled release tablets. In US 2003/0104049 Application, the problem linked to an excessive large size of the metformin tablets is solved deliberately excluding any use of a lubricant agent, such as magnesium stearate.

PCT Application WO 03/039527 solves the problem of the tablet large size resulting from the metformin low compressibility by combining (i) a non ionic hydrophilic polymer, such as a hydroxypropylmethylcellulose is with a molecular weight between 180,000 and 250,000 with (ii) an anionic hydrophilic polymer, such as sodium carboxymethylcellulose.

U.S. Pat. No. 6,117,451 solves the production problem of metformin based tablets by providing a single direct compression step of a complex combination of metformin hydrochloride and at least eight excipients, including hydroxypropylmethylcellulose, hydroxypropylcellulose, dibasic calcium phosphate and colloidal silicon dioxide. According to a main feature of the tablets disclosed in said patent, mefformin hydrochloride is incorporated under the form of particles with a size ranging from 70 μm to 110 μm, a feature without which it is not possible to perform the direct compression step. The tablets produced according to this patent teaching would have a short disintegration time, although no qualitative or quantitative data relating to the dissolution profile of the active substance are given.

Generally, even if there are satisfactory methods allowing to produce, from various adapted combinations of metformin and excipients, tablets based on such an active ingredient having the desired mechanical properties, the technical problems of producing tablets having a balance of the mechanical properties of (i) good preservation of the physical integrity of the tablets during the storage period and of (ii) fast disintegration of such tablets in contact with an aqueous solution at the time of use have never been completely overcome.

Thus, the metformin based tablets being commercially available today have good storage properties. On the contrary, such known tablets are disintegrated with difficulty during their use and none of them possess the properties of orodispersible and dispersible tablets laid by regulation in force.

Consequently, all metformin based tablets, being commercially available today, result, during their oral administration, in a high discomfort for the patients, more particularly for old-aged patients, accounting for more than 60% of the treated patients, for children, as well as for patients suffering from a buccopharyngeal disease. Practically, the patients usually have to manually disintegrate at least part of the tablet, for example, by grinding the tablet with the help of cutlery or a glass bottom, so as to form a coarse tablet powder, prior to ingesting the drug.

In order to overcome the general problems associated with administration of conventional metformin based tablets as described hereinabove, it has been suggested to substitute the solid pharmaceutical form for pharmaceutical compositions in a liquid form, which can be more easily administered, as disclosed in PCT Application WO 02/11716. However, the cost price of such a formulation in a liquid form is higher than that of the tablet form pharmaceutical composition. Moreover, the liquid volume to be administered is high. In addition, it is known that the liquid pharmaceutical compositions are much less stable than the tablet compositions. Finally, if administering a liquid formulation proves to be more comfortable for the patient, the compliance of a liquid formulation, i.e. the patients' adhesion to the prescription thereof is not improved, compared to the known tablet formulations.

There is consequently a need, in the state of the art, for galenic metformin based formulations, which would enable to solve the technical disadvantages associated with the known formulations, whether they are in a solid or liquid form.

### DETAILED DESCRIPTION OF THE INVENTION

The above-described technical disadvantages for the various known metformin based formulations are from now on solved according to the present invention, having as an object novel dispersible solid formulations, more particularly hydrodispersible and orodispersible formulations with an immediate release of such an active ingredient.

A metformin based solid pharmaceutical composition has been developed, according to the invention, allowing the production of non coated and non film-coated tablets having good storage properties, disintegrating in a very short time in contact with an aqueous solution, including water and saliva, and allowing for a fast release of the active ingredient after an oral administration or a prior dispersion in an aqueous solution. More specifically, the pharmaceutical composition which has been developed allows for mefformin based tablets to be produced which disintegrate after an immersion time in water lower than 3 minutes, as measured according to the standard from European Pharmacopeia (4th edition). This immediate disintegration effect is obtained more particularly by virtue of using, for producing the tablets of the invention, granules of the pharmaceutical composition with a size lower than 710 μm.

The features of the above-mentioned final hydrodispersible and orodispersible tablet have been reached according to the invention by developing a particular combination of the active ingredient(s) and excipients, as defined in the present specification.

According to the general monographs from European Pharmacopeia, a dispersible or hydrodispersible tablet consists in either a non coated or a film-coated tablet, adapted to be dispersed in water before being administered, resulting in a homogeneous dispersion (European Pharmacopeia, Section 4.4, pp. 3646). An orodispersible tablet is a non coated tablet adapted to be placed into the mouth where it quickly disperses before being swollen (European Pharmacopeia, Section 4.4, pp. 3646).

An orodispersible tablet disintegrates or breaks up in water R at 37° C. within less than 3 minutes.

A dispersible tablet disintegrates or breaks up in water R at 15° C.-25° C. within less than 3 minutes.

Moreover, a dispersible tablet breaks up resulting in dispersed particles, none of which has a size higher than 710 μm.

In order to determine the disintegration or break up time of a pharmaceutical composition according to the invention, after being shaped as dispersible tablets, the procedure is carried out as in the test referenced as “2.9.1” described in European Pharmacopeia (4^{th} Edition).

As indicated hereinabove, the dispersible tablets break up in into their component particles within less than three minutes in water R according the “2.9.1” test from European Pharmacopeia (4th Edition).

In addition, when two dispersible tablets according to the invention are immersed in 100 ml water R, and they are stirred until the total dispersion of the particles being contained in such tablets, the thus obtained particle dispersion is homogeneous and wholly goes through a sieve with a nominal mesh opening of 710 μm (European Pharmacopeia, 4^{th} Edition, Section 4-4).

As this will be detailed later in the specification, the tablets according to the invention do not include any coating or film-coating. Moreover, the tablets produced from the pharmaceutical composition according to the invention have, for a given metformin dosage, an identical, preferably lower, size compared to the previously known tablets.

In particular, it has been shown that the objects of the invention are reached for a pharmaceutical composition having a total weight not exceeding 1.6 times the metformin total weight contained in the latter, optionally presented under the form of one of the salts thereof, because of the use of appropriate relative amounts of binding agent(s) and disintegrating agent(s), for producing tablets without any film-coating or coating agent.

The invention has therefore as an object to provide a fast release dispersible and orodispersible solid pharmaceutical composition having the form of particles with a size lower than 710 μm, containing the metformin active ingredient, characterized in that it comprises:

a) from 65% to 90% in weight of the metformin active ingredient, optionally under the form of a salt, or a combination of the metformin active ingredient with a hypoglycemic active ingredient;

b) from 0.5 to 4% in weight of a binding agent or a combination of binding agents;

c) from 1% to 12% in weight of a disintegrating agent or a combination of disintegrating agents;

d) from 0% to 10% in weight of a diluting agent or a combination of diluting agents;

e) from 0.05% to 3% in weight of a sweetening agent or a combination of sweetening agents; and

f one or more additional excipients,

the weight percentages being expressed based on the total weight of said composition.

Compared to the compositions known for producing tablets, the pharmaceutical composition according to the invention comprises at least one sweetening agent, which tends, in nature, to considerably improve such a composition compliance, given the strong bitterness of the mefformin active ingredient.

Thus, another important feature of the pharmaceutical composition according to the invention is the presence, in such a composition, of an appropriate amount of at least one sweetening agent so as to mask the mefformin strong bitter taste. The pharmaceutical composition according to the invention may comprise a combination of two, three or four sweetening agents, provided that the weight percentage of the sweetening agent combination ranges from 0.005% to 3% based on the composition total weight.

In order to achieve the organoleptic effect of the sweetening agent or the combination of sweetening agents, the pharmaceutical composition according to the invention advantageously also comprises one flavouring agent or a combination of flavouring agents.

Thus, according to a preferred embodiment of the pharmaceutical composition according to the invention, the organoleptic features are further improved adding a flavouring agent or a combination of flavouring agents.

Thus, according to a first particular embodiment, the pharmaceutical composition according to the invention is characterized in that it also comprises 0.01% to 6% in weight of a flavouring agent, or a combination of flavouring agents, based on the composition total weight.

Preferably, the binding agent(s) is/are selected amongst polyvinylpyrrolidone, sodium carboxymethylcellulose, alginic acid, hydroxypropylmethylcellulose and polyethylene oxide.

As for polyvinypyrrolidone, a water-soluble polyvinylpyrrolidone is preferably selected with a molecular weight ranging from 44,000 to 54,000 (for example Kollidon® 30) or also those marketed under the trade names Kollidon® 25, Kollidon® 90 F, Plasdone® K-29/32, Plasdone® K-90 D/M, Povidone® K-90.

As for sodium carboxymethylcellulose, it is preferably selected amongst those marketed under the trade names Blanose®, Akucello®, Nymcel®.

As for hydroxypropylmethylcellulose, it is preferably selected amongst those marketed under the trade names Meocel®ES, Metolose.

As for polyethylene oxide, the one marketed under the designation Polyox® WSR N-10 is preferably selected.

Preferably, the disintegrating agent(s) is (are) selected amongst sodium croscarmellose, cross-linked polyvinylpyrrolidone, sodium starch glycolate, wheat or corn starch and pre-gelatinized starch.

As for sodium croscarmellose, it is preferably selected amongst those marketed under the trade names AC6Di-SOL®, Pharmacel XL, Primellose®, Solutab®, Nymcel ZSX.

As for cross-linked polyvinylpyrrolidone, it is preferably selected amongst those marketed under the trade names Kollidon® CL, Kollidon® CL-M, Polyplasdone® XL, Polyplasdone® XL-10, Polyplasdone® INF-10.

As for sodium starch glycolate, it is preferably selected amongst those marketed under the trade names Explotab®, Primopel®.

As for starch, corn starch is preferably selected.

As for pre-gelatinized starch, it is preferably selected amongst those marketed under the trade names Lycatab® C or Lycatab® PGS or C*Pharm® DC 93000; Starch®1500 as well.

Preferably, the diluting agent(s) is (are) selected amongst lactose, mannitol, cellulose, microcrystalline cellulose and calcium carbonate.

As for microcrystalline cellulose, it is preferably selected amongst those marketed under the trade names Vivapur® 99, Vivapur® 101, Vivapur® 102, Vivapur® 200, Avicel® PH 101, Avicel® PH 102, Avicel® PH 105, Avicel® PH 200, Tabulose® 101, Tabulose® 102, Tabulose® 250, Vivapur® 12, Vivapur® 20, Vivapur® 301, Vivapur® 302, Avicel® PH 112, Avicel® PH 113, Avicel® PH 301, Avicel® PH 302, Avicel® PH 103.

Preferably, the sweetening agent(s) is (are) selected amongst gluconate, aspartame, cyclamate, sodium saccharinate, xylitol and maltitol.

Preferably, the flavouring agent(s) is (are) selected amongst fruit flavour, mint flavour, anise flavour, honey flavour, vanilla flavour, tea flavour and verbena flavour. According to an important feature, the flavouring agent(s) included in the pharmaceutical composition according to the invention do not have any effect on glycemia.

More particularly, the following flavouring agent(s) are used apricot, orange-apricot, citrus fruit, pineapple-coconut, anise, banana, cocoa, caramel, fruit-caramel, blackcurrant, cherry, Morello cherry, raspberry-cherry, lemon, lime, orange essence, orange blossom, strawberry, raspberry, passion fruit, forest fruit, orchard fruit, red fruit, red fruit/caramel, grenadine, red currant, orange juice, mandarin, mango, mint, peppermint, eucalyptus-mint, honey, cherry plum, blackberry., bilberry, grape-fruit, peach, pear, apple, plum, orange pulp, grape, liquorice, orange tree-rosemary, tea, vanilla, verbena or violet.

Preferably, the flavouring agent is adsorbed onto an appropriate carrier and then incorporated into a pharmaceutical composition according to the invention, under the form of a previously impregnated powder of the carrier. Any carrier type conventional in the pharmaceutical field may be used as the flavouring agent, such as, for example, silica, starch or cellulose powder.

In a pharmaceutical composition according to the invention, the metformin or the metformin salt has advantageously the form of solid particles with a grain size lower than 100 μm. Using a metformin with a grain size lower than 100 μm allows for the final production of tablets using a simple and fast method essentially comprising a direct compression step of the pharmaceutical composition such as defined hereinabove, in the presence of an appropriate amount of a lubricant agent. Preferably the appropriate amount of the lubricant agent ranges from 0.01% to 1% in weight based on the total composition.

“Grain size” of an immediate release micronized powder according to the invention means the average size of the component grains. The grain average size can be measured using any conventional technique known per se. More particularly, the man of the art may use a laser measurement of the grain size using a Beckman Coulter® or Malvern®, as described in the examples.

The metformin salts are preferably selected amongst phosphate, sulfate, hydrochloride, salicylate, maleate, benzoate, ethanedisulfonate, fumarate, succinate, chlorophenoxyacetate, embonate and glycolate salts.

Using 0.5% to 3.5% in weight of the binding agent or the combination of binding agents allows to efficiently binding to each other the granules of active ingredient, which is itself practically free of any binding power.

In addition, using 1% to 12% in weight of a disintegrating agent or a combination of disintegrating agents allows for contributing essentially to the disintegration mechanical features in an aqueous solution of the tablets which are subsequently produced.

All the disintegrating agents specified in the present specification may be implemented for producing tablets having the quality of dispersible and orodispersible tablets, which have good storage properties and the required properties of quick disintegration in the constituent particles, after contact with water or an aqueous solution.

However, some disintegrating agents, such as polyvinylpyrrolidone, in particular polyvinylpyrrolidone with a molecular weight ranging from 44,000 and 54,000, are those that make it possible to obtain the best results.

In particular, it has been shown that when a disintegrating agent has been used such as pre-gelatinized or partially pre-gelatinized corn starch, for example Lycatab® C or Lycatab® PGS, the tablets which are then produced, although meeting requirements of the invention, have mass and hardness irregularities. Such an effect is more particularly shown for the composition according to the invention described in the examples 5, 6, and 8 to 10.

Moreover, a disintegrating agent amount higher than that prescribed leads to an unacceptable longer release time for metformin. This is the reason why using a disintegrating agent in weight percentages higher than 12% should be avoided. In order to obtain the best results, it would be advantageous to use an amount of the disintegrating agent or the combination of disintegrating agents that does not exceed 6% in weight, based on the composition total weight.

Also, too high a proportion of diluting agent could lead to disadvantages relating to the storage properties, the disintegration properties during contact with water, and the release properties of the active ingredient(s). Preferably, the diluting agent amount should not be higher than 8% in weight, based on the composition total weight.

In order to complete the hypoglycemic or normoglycemic effect of the metformin, for treating the patient, the pharmaceutical composition according to the invention comprises, in association with metformin, also a second hypoglycemic active ingredient, amongst the known hypoglycemic active ingredients, which are active at low doses, such as glicazide, glipizide, chlorpropamid, glimepiride glibenclamide and derivatives thereof.

Alternatively, the pharmaceutical composition according to the invention can comprise, in association with metformine, a second active ingredient selected amongst:
a PPAR Gamma agonist (peroxisome proliferator-activated receptor gamma) or Glitazone, like rosiglitazone, pioglitazone, and balaglitazone and derivatives thereof, or
a PPAR Gamma and Alpha agonist or Glitazar like terapglitazar, muraglitazar, and ragaglitazar and derivatives thereof, or
a dipeptidyl peptidase inhibitor (DPPIV), or
acarbose or derivative thereof, or
a hypocholesterol agent of fibrate type, such as fenobibrate and derivatives thereof.

In a pharmaceutical composition according to the invention, the second hypoglycemic active ingredient is present, preferably, in an amount ranging from 0.01% to 10% in weight, based on the composition total weight, in the combination from 65% to 90% in weight of the active ingredient association.

According to a particular embodiment of the pharmaceutical composition of the invention, the latter is characterized in that it comprises:

a) from 65% to 80% in weight of the metformin active ingredient, optionally under the form of a salt, or a combination of the metformin active ingredient avec a hypoglycemic active ingredient;

b) from 0.5 to 4% in weight of a water-soluble polyvinylpyrrolidone having a molecular weight ranging from 44,000 to 54,000;

c) from 1% to 10% in weight of a water-insoluble cross-linked polyvinylpyrrolidone;

d) from 0.5% to 10% in weight of a diluting agent or a combination of diluting agents;

e) from 0.05% to 3% in weight of a sweetening agent or a combination of sweetening agents; and

f) one or more additional excipients

the weight percentages being expressed based on the total weight of said composition.

Most preferably, in order to prepare tablets with the optimum mechanical and disintegrating features, the pharmaceutical composition according to the invention, after disintegration in water, does not comprise any particle from the disintegration having a size higher than 710 μm. It is understood that each granule generated from the tablet disintegration consists in (i) an internal core comprising the active ingredient in association with the appropriate excipients, and (ii) an external layer comprising the sweetening agent in association with the appropriate excipient(s).

Advantageously, in order to reach the mechanical features and the immediate release features of the active ingredient which are desired, the internal core accounts for 75% to 85% in weight, based on the composition total weight and the external layer accounts for 15% to 25% in weight, based on the total weight of said composition.

Most preferably, the metformin is included in the internal core in association with the binding agent, preferably the water-soluble polyvinylpyrrolidone with a molecular weight ranging from 44,000 and 54,000; the core may additionally comprise, in some embodiments, also one or more appropriate excipients, mainly one or more other binding agents and, in some embodiments, one or more binding agents and, in some embodiments, also a sweetening agent or a combination of sweetening agents as well as, if need be, a flavouring agent or a combination of flavouring agents.

Most preferably, the external layer comprises excipients which will impart to the tablets to be produced their mechanical features and the active ingredient release features, i.e. essentially the disintegrating agent or the combination of disintegrating agents, preferably the water-insoluble cross-linked polyvinylpyrrolidone. Also in the external layer is included the sweetening agent or the combination of sweetening agents. Preferably, the flavouring agent or the combination of flavouring agents is also included in the external layer. Finally, when the tablet is being produced, an appropriate amount of a lubricant agent or a combination of lubricant agents is added to the external layer.

The lubricant agent(s) is (are) preferably selected amongst magnesium stearate, stearic acid and the derivatives thereof, sodium stearyl fumarate and sodium benzoate.

Thus, according to a preferred embodiment of the granules of the pharmaceutical composition of the invention, the whole metformin is included into the internal core of said granules.

Another object of the invention is also a pharmaceutical composition such as previously defined, said composition consisting respectively in:

(i) an internal core comprising:

a) from 65% to 80% in weight of the metformin active ingredient, optionally under the form of a salt or a combination of the metformin active ingredient with a hypoglycemic active ingredient; and

b) from 0.5% to 4% in weight of a binding agent or a combination of binding agents; and

(ii) an external non film-coated layer comprising:

a) from 0% to 10% in weight of a diluting agent or a combination of diluting agents;

b) from 1% to 10% in weight of a disintegrating agent or a combination of disintegrating agents; and

c) from 0.05% to 3% in weight of a sweetening agent or a combination of sweetening agents;

the weight percentages being expressed based on the total weight of said composition.

Preferably, the binding agent is a water-soluble polyvinylpyrrolidone with a molecular weight ranging from 44,000 to 54,000.

Preferably, the disintegrating agent is a water-insoluble cross-linked polyvinylpyrrolidone.

The invention also relates, in one of its preferred embodiments, to a pharmaceutical composition such as previously defined, said composition comprising:

(i) an internal core comprising:

a) from 76% to 77% in weight of the metformin chlorhydrate active ingredient, optionally under the form of a salt; and

b) from 2.5% to 3.5% in weight of a water-soluble polyvinylpyrrolidone with a molecular weight ranging from 44,000 to 54,000; and

(ii) an external non film-coated layer comprising:

a) from 6.5% to 7.5% in weight of a diluting agent or a combination of diluting agents;

b) from 4.5% to 5.5% in weight of a water-insoluble cross-linked polyvinylpyrrolidone; and

c) from 0.5% to 2.5% in weight of a sweetening agent or a combination of sweetening agents;

the weight percentages being expressed based on the total weight of said composition.

The present invention also relates to a pharmaceutical composition such as defined hereinabove, characterized in that it consists in:

(i) an internal core comprising:

a) 76.92% in weight of the metformin hydrochloride active ingredient; and

b) 3.08% in weight of a water-soluble polyvinylpyrrolidone with a molecular weight ranging from 44,000 and 54,000; and

(ii) an external non film-coated layer comprising:

a) 7% in weight of a diluting agent or a combination of diluting agents;

b) 5% in weight of a water-insoluble cross-linked polyvinylpyrrolidone;

c) 2% in weight of a sweetening agent or a combination of sweetening agents;

d) 5% in weight of a flavouring agent or a combination of flavouring agents; and

e) 1% in weight of a preservative;

the weight percentages being expressed based on the total weight of said composition.

As already been mentioned hereinabove, the metformin preferably has the form of a salt selected amongst phosphate, sulfate, hydrochloride, salicylate, maleate, benzoate, ethanedisulfonate, fumarate, succinate, chlorophenoxyacetate, embonate and glycolate salts

The pharmaceutical composition such as described in detail hereinabove is used for producing metformin based hydrodispersibles tablets, according to any of the various tablet production methods known in the state of the art.

Another object of the invention is therefore also to provide a hydrodispersible non film-coated and a non coated pharmaceutical tablet, characterized in that it comprises a pharmaceutical composition such as defined hereinabove.

It has been shown that the tablets produced from a pharmaceutical composition according to the invention could be stored for several months, without any important change in their mechanical, hardness features, as opposed to numerous previously known metformin formulations in tablet forms. Thus, with tablets prepared using known pharmaceutical compositions, an important increase is observed of the tablet hardness during the storage time. More particularly, with some conventional tablets with an initial hardness, after production, in the order of 100 N, the hardness optionally may increase up to 500 N after several storage months, which would force the patient to grind the tablet so as to obtain a tablet coarse powder that could be orally ingested, optionally after a preliminary suspension in water or in any aqueous solution. Indeed, the increase over the time of the hardness of the conventional tablets simultaneously leads to an important degradation of the disintegration features of such tablets, which, consequently, are not hydrodispersible or orodispersible tablets, according to the definition of the European Pharmacopeia. On the contrary, the specific combination of active ingredient(s) and excipients of the pharmaceutical composition according to the invention, especially when it is a pharmaceutical composition under the form of granules each having the previously described internal core and external layer, allows for the production of tablets whose hardness features do not vary during the storage period, which, consequently, when being used by the patient, have the features required by European Pharmacopeia to be referred to as dispersible tablets, in particular, hydrodispersible and orodispersible ones.

Preferably, each tablet according to the invention comprises an amount of metformin, optionally present in the form of one of its salts, ranging from 100 mg to 3000 mg, advantageously from 250 mg to 1200 mg, preferably from 250 mg to 1000 mg. A tablet according to the invention may also contain an amount of metformin, optionally present in the form of a salt, of 250 mg, 500 mg, 750 mg, 850 mg, 1000 mg or 1100 mg.

According an essential feature, because of the specific qualitative and quantitative combination in active ingredient and excipients of the above-defined pharmaceutical composition, it is not required to coat a metformin tablet according to the invention with any external coating or film-coating layer.

More particularly, a coating or a film-coating intended to mask the bitterness of the metformin is not necessary, because of the presence of one or more sweetening agents and optionally also of the flavouring agent(s).

Secondly, given the good mechanical features and the good release features of the active ingredient of the tablets produced from the pharmaceutical composition of the invention, a protective coating or film-coating would be a disadvantage, as it would be subject to modify said mechanical features or said release profile features of the active ingredient. Indeed, using a coating or film-coating agent would have the effect of considerably increasing the disintegration time of the tablet into its constituent granules, in contact with water or an aqueous solution.

Advantageously, a tablet according to the invention has a breaking strength higher than 100 N and may be dispersed in distilled water at 20° C. within less than 10 minutes, more preferably within less than 5 minutes or most preferably within less than 3 minutes.

Advantageously, a tablet according to the invention has a breaking strength higher than 110 N and more preferably higher than 120 N.

Quite preferably, a tablet according to the invention may be dispersed in distilled water at 20° C. within less than 2 minutes, more preferably within less than 1.5 minutes and quite preferably within less than 1 minute.

It has been shown according to the invention that the method described above makes it easy and less expensive to obtain tablets of mefformine or one of its derivatives, optionally in combination with a second active ingredient, which possess an in vitro pharmacokinetic profile and an in vivo pharmacokinetic profile which is at least identical with that observed with the tablets of metformine prepared according to the methods described in the prior art.

Maximum plasma concentration values (Cₘₐₓ), time values until the maximum plasma concentration has been reached (Tₘₐₓ) and AUC values (for the area under the plasma concentration curve) have been calculated from pharmacokinetic profiles for tablets prepared according to the invention and for immediate release tablets (non dispersible) prepared according to a method comprising a wet granulation step, such as in the prior art. All these values have been compared in examples 14.

Preferably the tablet according to the invention exhibits a pharmacokinetic profile established from two tablets, each dosed at 500 mg, characterized by an area under the plasma concentration curve measured in vivo (AUC) ranging from 10000 ng.h/ml to 16250 ng.h/ml and preferably of about 12500 ng.h/ml

Preferably the tablet according to the invention exhibits a pharmacokinetic profile established from two tablets, each dosed at 500 mg, characterized by a maximum plasma concentration value (Cₘₐₓ) ranging from 1600 ng/ml to 2600 ng/ml and preferably of about 2000 ng/ml.

Preferably the tablet according to the invention exhibits a pharmacokinetic profile established from two tablets, each dosed at 500 mg, is characterized by a Tₘₐₓ value ranging from 2 h and 3.25 h and preferably of about 2.5 h.

It will be appreciated by the one skilled in the art that “Tₘₐₓ” as used herein corresponds to the time until the maximum plasma concentration has been reached.

The tablet according to the invention, dosed at 500 mg in metformine chlorhydrate, gives some results that can be extrapolated to the compositions of the same type and comprising lower doses in mefformine chlorhydrate, metformine chlorhydrate absorption in human beings, being linear from 0 to 1000 mg.

Preferably, the tablet according to the invention, dosed at 500 mg, releases between 50% and 100% of the active ingredient dose and preferably at least 80% of the metformine chlorhydrate dose in 5 minutes in a physiological buffer medium pH 6,8 at 37° C.

The tablets according to the invention, dosed at 500 mg in metformine chlorhydrate, taken in two separate doses of 500 mg give some results that can be extrapolated to the compositions of the same type and comprising lower doses in metformine chlorhydrate, metformine chlorhydrate absorption in human beings, being linear from 0 to 1000 mg.

The extrapolation for AUC and Cₘₐₓ can be done on the basis of a rule of three, the Tₘₐₓ value staying unchanged, ranging from 2 h and 3.25 h and preferably of about 2.5 h.

The qualitative and quantitative composition in active ingredient and excipients of the pharmaceutical composition according to the invention allows for the production of tablets, from such a composition, according to various methods, respectively dry granulation or wet granulation methods, or direct compression methods as well.

For implementing a method for producing tablets comprising a granulation step, whether dry or wet granulation, metformin or its salt in the granule form is used, with a grain size lower than 100 μm. The granulation step allows for increasing the density of the core containing the active ingredient.

As is conventional, in order to produce a tablet according to the invention, at the very end, an appropriate amount of a lubricant agent or a combination of lubricant agents is added to the granules, before the final compression step, so as to minimize the adhesion phenomenon of the tablets at the punch surface.

Thus, another object of the invention is also a method for preparing a pharmaceutical tablet such as defined hereinabove, characterized in that it comprises the following steps of:

a) preparing the core (i) such as previously defined, through wet granulation of a mixture of metformin appropriate amounts, optionally under the form of a salt, and water-soluble polyvinylpyrrolidone, having a molecular weight ranging from 44,000 to 54,000;

b) drying the granules obtained in step a);

c) adding to the granules obtained in step b) the excipient mixture forming the external layer (ii) such as previously defined; and

d) performing a compression of the granules obtained in step c).

The invention also relates to a method for preparing a pharmaceutical tablet such as hereinabove defined, characterized in that it comprises the following steps of:

a) preparing the core (i) such as previously defined, through dry granulation of a mixture of metformin appropriate amounts, optionally under the form of a salt, and water-soluble polyvinylpyrrolidone, having a molecular weight ranging from 44,000 to 54,000;

b) compacting the dry granules obtained in step a);

c) adding to the granules obtained in step b) the excipient mixture forming the external layer (ii) such as previously defined; and

d) performing a compression of the granules obtained in step c).

The present invention also relates to a method for preparing a pharmaceutical tablet such as defined hereinabove, characterized in that it comprises the following steps of:

a) preparing a mixture of the core (i) constituents such as defined hereinabove, through dry granulation of a mixture of the metformin appropriate amounts, optionally under the form of a salt and water-soluble polyvinylpyrrolidone soluble having a molecular weight ranging from 44,000 to 54,000;

b) adding to the granules obtained in step a), the excipient mixture forming the external layer (ii) such as defined hereinabove; and

c) performing a compression of the granules obtained in step b).

The latter method for producing tablets is preferably used when the metformin or the salt thereof has the form of particles with a grain size higher than 100 μm.

The present invention is also illustrated, without being thereto limited, by the following examples.

### FIGURES

FIG. 1 illustrates a comparative study of the in vitro dissolution profile of metformine tablets dosed at 500 mg according to the invention, varying upon time. 3 graphs (start, middle, end of compression) are presented and shows the time when the tablet from the 23421 batch have been submitted to the compression step.

This study has been carried out according to the following in vitro dissolution parameters:
dissolution apparatus with rotary pales SOTAX AT 7 (or equivalent)
Lambda spetrophotometer 20 PERKIN ELMER (or equivalent)
Medium: 500 ml of physiological buffer solution pH 6.8
Wave length 260 nm
Quartz tank with optical length of 1 cm
pales rotation speed: 75 rpm

FIG. 2 illustrates a comparative study of the in vitro dissolution profile of mefformine tablets dosed at 1000 mg according to the invention, varying upon time. 3 graphs (start, middle, end of compression) are presented and shows the time when the tablet from the 23422 batch have been submitted to the compression step.

This study has been carried out according to the following in vitro dissolution parameters:
dissolution apparatus with rotary pales SOTAX AT 7 (or equivalent)
Lambda spetrophotometer 20 PERKIN ELMER (or equivalent)
Medium: 500 ml of physiological buffer solution pH 6.8
Wave length 260 nm
Quartz tank with optical length of 1 cm
pales rotation speed: 75 rpm

FIG. 3 illustrates a comparative study of the in vivo pharmacokinetic profile between orodispersible or dispersible metformine tablets dosed at 500 mg according to the invention, given in to separate doses of 500 mg, and film-coated metformine tablets dosed at 500 mg administered in two separate doses of 500 mg, marketed under the trade mark Glucophage®

FIG. 4 is a napierian logarithmic (Ln) view of the graphs from FIG. 3.

### EXAMPLES

### Example 1

### Metformin HCl 1000 mg Dispersible Composition—Wet Granulation Method

1. Ingredients

metformine HCl
Povidone K30 (KOLLIDON® 30)
Crosspovidone (KOLLIDON® XL)
grapefruit-orange flavour
aspartame
magnesium stearate
   2. Operation Mode
   The metformine HCl is introduced into a high speed granulating mixer of the ROTOLAB® type.
   The povidone is solubilized in purified water (25% m/m).
   The granulation step occurs in the ROTOLAB® equipped with a three-bladed type stirring axis and through incorporation of the povidone solution on the active ingredient. The step is achieved by a sufficient amount of water so as to obtain a satisfactory quality grain.
   The thus obtained grain is dried in a fluidized air bed of the Mini GLATT type for about 5 minutes at 400° C. so as to obtain a grain with a residual humidity close to 1.0%.
   The grain is calibrated using an oscillating granulator of the ERWEKA type equipped with a stainless steel grid with a 1,0 mm mesh opening diameter.
   The calibrated grain is introduced into a tumbling powder mixing vessel of the TURBULA type or equivalent.
   The flavour, the sweetener, and the disintegrating agent are added to the mixture.
   The mixing time is set to approximately 10 minutes.
   The lubricant agent is added to the mixture and the mixing time is approximately 1 minute.
   The thus obtained final mixture is introduced into the feed hopper of the rotary pressing machine of the MR6 type equipped with a 20×9.5 forming punch.

3. Tablet Features

| | |
|---|---|
| average mass: | 1 100 mg |
| hardness: | 160 N |
| break up: | 53 seconds |
| particle fineness (<710 μm): | conform |

### Example2

### Metforming HCl 500 mg Dispersible Composition—Wet Granulation Method

1. Ingredients

metformin HCl
Copovidone (KOLLIDON® VA 64)
hydrated silica
orange flavour
citric acid
L-HPC 11
magnesium stearate
   2. Operation Mode
   The metformine HCl is mixed with the copovidone, hydrated silica, flavour, citric acid and L-HPC 11 for 10 minutes using the TURBULA.
   The mixture is then compacted, and then calibrated to 3.5 mm and then 1.0 mm. The granule is then mixed with the magnesium stearate for about 3 minutes.
   The final mixture is then compressed on a rotary pressing Is machine of the MR6 type equipped with a 20×9,5 forming punch.
   A fraction of the final mixture is also compressed on 12R16 size punches.

3. Tablet Features

| | |
|---|---|
| average mass: | 1 200 mg |
| hardness: | 250 N |
| break up: | 1 mn 30 |
| particle fineness(<710 μm): | conform |

### Example3

### Metformin HCl 1000 mg Dosed Dispersible Composition and mg of Glipizide Produced Through Wet Granulation

1. Ingredients

metformine HCl
glipizide
Povidone K30 (KOLLIDON® 30)
Crosspovidone (KOLLIDON® XL)
grapefruit-orange flavour
aspartame
magnesium stearate
   2. Operation Mode
   The metformin HCl and glipizide are introduced into a high speed granulator mixer of the ROTOLAB® type.
   The povidone is solubilized in purified water (25% m/m).
   The granulation operation is carried out in the ROTOLAB® equipped with a three-bladed type stirring axis and through the incorporation of the povidone solution onto the active ingredient. The operation is achieved with a sufficient amount of water so as to obtain a satisfactory quality grain.
   The thus obtained grain is dried in a fluidized air bed of the Mini GLATT type for about 5 minutes at 40° C. so as to obtain a grain with a residual humidity close to 1.0%.
   The grain is calibrated using an oscillating granulator of the ERWEKA type equipped with a stainless steel grid with a 1.0 mm mesh opening diameter.
   The calibrated grain is introduced into a tumbler powder mixing vessel of the TURBULA type or equivalent.
   The flavour, the sweetener, and the disintegrating agent are added to the mixture.
   The mixing time is set to approximately 10 minutes.
   The lubricant agent is added to the mixture and the mixing time is approximately 1 minute.
   The thus obtained final mixture is introduced into the feed hopper of the rotary pressing machine of the type MR6 equipped with a 20×9.5 forming punch.

3. Tablet Features

| | |
|---|---|
| average mass: | 1 100 mg |
| hardness: | 160 N |
| break up: | 53 seconds |
| particle fineness(<710 μm): | conform |

### Example 4

### Metformin HCl 1000 mg Dispersible Composition—Wet Granulation Method

1. Ingredients

metformine HCl
Povidone K30 (KOLLODON®30)
Crosspovidone (KOLLIDON® XL)
grapefruit-orange flavour
xylitol 10 (70% m/m)
sodium saccharinate 0.1% (1% m/m)
magnesium stearate
   2. Operation Mode
   The metformin HCl is introduced in a high speed granulator mixer of the ROTOLAB® type.
   The povidone is solubilized in purified water (25% m/m).
   The granulation step is performed in the ROTOLAB® equipped with a three-bladed type stirring axis and through the incorporation of the povidone solution on the active ingredient. The step is achieved by a sufficient amount of water so as to obtain a satisfactory quality grain.
   The thus obtained grain is dried in a fluidized air bed of the Mini GLATT type for about 5 minutes at 40° C. so as to obtain a grain with a residual humidity close to 1.0%.
   The grain is calibrated using an oscillating granulator of the ERWEKA type equipped with a stainless steel grid with a 1.0 mm mesh opening diameter.
   The calibrated grain is introduced into a tumbling powder mixing vessel of the TURBULA type or equivalent
   The flavour, the sweetener, and the disintegrating agent are added to the mixture.
   The mixing time is set to approximately 10 minutes.
   The lubricant agent is added to the mixture and the mixing time is approximately 1 minute.
   The thus obtained final mixture is introduced into the feed hopper of the rotary pressing machine of the type MR6 equipped with a 20×9.5 forming punch.

3. Tablet Features

| | |
|---|---|
| average mass: | 1 100 mg |
| hardness: | 160 N |
| break up: | 53 seconds |
| particle fineness(<710 μm): | conform |

### Examples 5 to 10

The qualitative and quantitative composition in active ingredient and excipients of the compositions in the examples 5 to 10, as well as the s other studied features of such compositions, are detailed in table 1 hereinunder.

The pharmaceutical composition allowing to produce tablets having the best features, both from the point of view of hardness, disintegration time of the tablet in aqueous solution and the active ingredient release time, is the composition in example 7.

TABLE 1

| | Example 5 | Example 6 |
|---|---|---|
| Composition (amounts in %) | GAL 332-01 | GAL 332-02 |
| | 03CDA053103 | 03CV061101 |
| Internal phase | | |
| A0130 | 76.92 | 76.92 |
| Powder Kollidon 30 | 2.08 | 2.08 |
| Kollidon 30 (20% solution) | 1.00 | 1.00 |
| Lycatab C | — | — |
| External phase | | |
| Type 12 microcrystalline cellulose | 9.00 | 7.00 |
| (Vivapur) | | |
| Grapefruit orange flavour 35B250 | 4.00 | 5.00 |
| Sodium saccharinate | 1.00 | 2.00 |
| Micronized sodium benzoate | 1.00 | 1.00 |
| Crospovidone (Kollidon CL) | — | — |
| Lycatab C | 5.00 | 5.00 |
| Lycatab PGS | — | — |
| Theoretical weight of the tablets | 1300 | 1300 |
| (mg) | | |
| Drying mode | Steamroom | Steamroom |
| Calibration | 1 mm | 1 mm |
| Grain checks | 03CV052202 | 03CV060501 |
| Humidity (%) | 0.85 | 0.90 |
| Flow (sec) | 7.54 | 6.22 |
| Measurement of the bulk volume | | |
| Density before packing | 0.521 | 0.51 |
| Density after packing | 0.588 | 0.58 |
| Packing ability (ml) | 7 | 7 |
| Grain size analysis (centering in | 500 | 500 |
| μm) | | |
| Features | Granulated aspect, | Electrostatic, |
| | homogeneous, little | homogeneous size white |
| | electrostatic white powder | grain |
| Final mixture check | 03CV052602 | 03CV061001 |
| Humidity (%) | | 2.09 |
| Flow (s) | 2.47 for 50 g | 5.04 |
| Measurement of the bulk volume | | |
| Density before packing | | 0.57 |
| Density after packing | | 0.66 |
| Packing ability (ml) | | 10 |
| Grain size analysis (centering in | | 355 |
| μm) | | |
| Features | | Little electrostatic, |
| | | granulated aspect |
| | | powder, presence of |
| | | fines, |
| | | white/yellowish mixture. |
| | | Grapefruit/orange smell. |
| Punch size | 20 × 9.5 ALTERNATIVE | 20 × 9.5 ALTERNATIVE |
| | ELLIPSE | ELLIPSE |
| Finished product checks | 03CDA053101 | 03CV061101 |
| Humidity (%) | | |
| Average mass (mg) | | |
| Mass uniformity | | |
| Break strength (N) | 94 N | 129.85N |
| Dispersion | 31 s “flash” effect | 2 mn 22 s |
| Break up (distilled water 37° C.) | | 1 mn 57 s |
| Break up (distilled water 20° C.) | | 3 mn 00 s |
| Size checks: Thickness | | |
| (mm) | | |
| Diameter | | |
| (mm) | | |
| Friability (%) | | |
| Features | | |
| OBSERVATIONS | Improve the aromatization | Mass/hardness |
| | and the sweetening in | irregularity of the mixture - |
| | dispersion | new composition |

| | Example 7 | | Example 8 | |
|---|---|---|---|---|
| Composition (amounts in %) | GAL 332-03 | | GAL 332-04 | |
| | 03VG060601 | | 03VG060701 | |
| Internal phase | | | | |
| A0130 | 76.92 | | 76.92 | |
| Powder Kollidon 30 | 2.08 | | 2.08 | |
| Kollidon 30 (20% solution) | 1.00 | | 1.00 | |
| Lycatab C | — | | — | |
| External phase | | | | |
| Type 12 microcrystalline cellulose | 7.00 | | 7.00 | |
| (Vivapur) | | | | |
| Grapefruit orange flavour 35B250 | 5.00 | | 5.00 | |
| Sodium saccharinate | 2.00 | | 2.00 | |
| Micronized sodium benzoate | 1.00 | | 1.00 | |
| Crospovidone (kollidon CL) | 5.00 | | 2.50 | |
| Lycatab C | — | | 2.50 | |
| Lycatab PGS | — | | — | |
| Theoretical weight of the tablets | 1300 | | 1300 | |
| (mg) | | | | |
| Drying mode | Steamroom | | Steamroom | |
| Calibration | 1 mm | | 1 mm | |
| Grain checks | 03CV052202 | | 03CV052202 | |
| Humidity (%) | 0.85 | | 0.85 | |
| Flow (s) | 7.54 | | 7.54 | |
| Measurement of the bulk volume | | | | |
| Density before packing | 0.521 | | 0.521 | |
| Density after packing | 0.588 | | 0.588 | |
| Packing ability (ml) | 7 | | 7 | |
| Grain size analysis (centering in | 500 | | 500 | |
| μm) | | | | |
| Features | Little electrostatic, | | Little electrostatic, | |
| | homogeneous, granulated | | homogeneous, granulated | |
| | aspect white powder | | aspect white powder | |
| Composition (amounts in %) | GAL 332-03 | | GAL 332-04 | |
| | 03VG060601 | | 03VG060701 | |
| Final mixture checks | 03VG060401 | | 03VG060402 | |
| Humidity (%) | | | | |
| Flow (s) | 2.15 for 50 g | | 1.97 for 50 g | |
| Measurement of the bulk volume | | | | |
| Density before packing | | | | |
| Density after packing | | | | |
| Packing ability (ml) | | | | |
| Grain size analysis (centering in | | | | |
| μm) | | | | |
| Features | | | | |
| Punch size | 20 × 9.5 ALTERNATIVE | | 20 × 9.5 ALTERNATIVE | |
| | ELLIPSE | | ELLIPSE | |
| Finished product checks | 03VG060601 | | 03VG060701 | |
| Humidity (%) | | | | |
| Average mass (mg) | | | | |
| Mass uniformity | | | | |
| Break strength (N) | 136 N | | 114 N/95 N | |
| Dispersion | Dispersion approximately 1 min | | Dispersion >1 min/50 s | |
| Break up (distilled water 37° C.) | | | | |
| Break up (distilled water 20° C.) | | | | |
| Size checks: Thickness | | | | |
| (mm) | | | | |
| Diameter | | | | |
| (mm) | | | | |
| Friability (%) | | | Friable porous | |
| Features | | | | |
| OBSERVATIONS | | | | |
| | Example 9 | | | Example 10 |
| Composition (amounts in %) | GAL 332-05 | | | GAL 332-06 |
| | 03VG061202/03CV061201 | | | |
| Internal Phase | | | | |
| A0130 | 76.92 | | | 76.92 |
| Powder Kollidon 30 | 2.08 | | | 2.08 |
| Kollidon 30 (20% solution) | 1.00 | | | 1.00 |
| Lycatab C | — | | | — |
| External phase | | | | |
| Type 12 microcrystalline cellulose | 7.00 | | | — |
| (Vivapur) | | | | |
| Orange grapefruit flavour 35B250 | 5.00 | | | 5.00 |
| Sodium saccharinate | 2.00 | | | 2.00 |
| Micronized sodium benzoate | 1.00 | | | 1.00 |
| Crospovidone (kollidon CL) | — | | | — |
| Lycatab C | — | | | — |
| Lycatab PGS | 5.00 | | | 12.00 |
| Theoretical weight of the tablets | 1300 | | | 1300 |
| (mg) | | | | |
| Drying mode | Steamroom | | | Steamroom |
| Calibration | 1 mm | | | 1 mm |
| Grain checks | 03VG061102 | | | 03VG061102 |
| Humidity (%) | 0.94 | | | 0.94 |
| Flow (s) | 6.12 | | | 6.12 |
| Measurement of the bulk volume | | | | |
| Density before packing | 0.45 | | | 0.45 |
| Density after packing | 0.51 | | | 0.51 |
| Packing ability (ml) | 8 | | | 8 |
| Grain size analysis (centering in | 250 | | | 250 |
| μm) | | | | |
| Features | Powder-like, slightly electrostatic, | | | Powder like, |
| | homogeneous white grain | | | slightly |
| | | | | electrostatic, |
| | | | | homogeneous |
| | | | | white |
| | | | | grain |
| Final mixture checks | 03VG061201 | | | 03VR061302 |
| Humidity (%) | | | | |
| Flow (s) | 2.25 for 50 g | | | 4.50 |
| Measurement of the bulk volume | | | | |
| Density before packing | | | | |
| Density after packing | | | | |
| Packing ability (ml) | | | | |
| Grain size analysis (centering in | | | | |
| μm) | | | | |
| Features | | | | |
| Punch size | 20 × 9.5 ALTERNATIVE ELLIPSE | | | 20 × 9.5 |
| | | | | ALTERNATIVE |
| | | | | ELLIPSE |
| Finished product checks | 03VG061202 | 03CV061201 | | 03CV061701 |
| Humidity (%) | | | | |
| Average mass (mg) | | | | |
| Mass uniformity | | | | |
| Break strength (N) | 90N and 150N | 134N/113N | 113 N | 39N/74N/88N |
| Dispersion | 50 s at 1 min/>1 min | >1 min | >1 min | 2′02″/2′37″/2′50″ |
| Break up (distilled water 37° C.) | | 1 min 37 s | 1 min 37 s | |
| Break up (distilled water 20° C.) | | 2 min 19 s | 2 min 19 s | |
| Size checks: Thickness | | | | |
| (mm) | | | | |
| Diameter | | | | |
| (mm) | | | | |
| Friability (%) | Firable | | | |
| Features | | | | |
| OBSERVATIONS | | | | Too slow |
| | | | | dispersion |

### Example 11

### Dispersable Composition of Metformine HCl 500 mg and 1000 mg

The metformine tablets of two batches, dosed at 500 mg (batch 23421) and dosed at 1000 mg (batch 23422) have been prepared in conformity with the procedure disclosed in examples 1-4 from a same mix (batch 23419). The qualitative and quantitative concentrations in active ingredients and excipients of the tablets dosed at 500 mg and 1000 mg are presented in Table 2 below.

TABLE 2

| | Formula | | | Batch |
|---|---|---|---|---|
| | Percent | | | 23419 |
| Composition | (%) | (mg/tablet) | | (kg) |
| Active substance | | | | |
| Metformin | 76.92 | 500.00 | 1000.00 | 500.00 |
| Excipients | | | | |
| Povidone | 3.08 | 20.00 | 40.00 | 20.00 |
| Microcrystalline cellulose | 9.50 | 61.75 | 123.50 | 61.75 |
| Sodium saccharinate | 2.00 | 13.00 | 26.00 | 13.00 |
| Sodium benzoate | 1.00 | 6.50 | 13.00 | 6.50 |
| Pregelatinised starch | 5.00 | 32.50 | 65.00 | 32.50 |
| Lemon flavor | 2.50 | 16.25 | 32.50 | 16.25 |

| | | |
|---|---|---|
| Tablet theoretical weight (mg) | 650.00 | 1300.00 |

The tablets obtained have to satisfy the requirements shown in table 3 below.

TABLE 3

Specifications

| Active substance | 500 mg | 1000 mg |
|---|---|---|
| Awl size | 12R14 | 19 × 10 |
| Average mass | 650.0 mg +/− 3% | 1300.0 mg +/− 3% |
| Mass uniformity | Comply with | Comply with |
| | Eur. Ph (2.9.5) | Eur. Ph. (2.9.5) |
| Subdivision | — | Comply with |
| | | Eur. Ph. (2.9.5) |
| Hardness | 40 to 80 N | 100 to 140 N |
| Flakiness | ≦1.0% | ≦1.0% |
| Break up (20° C.) | ≦3 min | ≦3 min |
| Break up (37° C.) | ≦3 min | ≦3 min |
| Dispersion fineness | <710 μm | <710 μm |

a) Pharmocotechnical Features of the Grain Obtained

The pharmocotechnical features of the grain are shown in table 4 below.

TABLE 4

| TEST | | Batch 23419 | |
|---|---|---|---|
| Flow (100 g) | | 4.92 | sec |
| Bulk volume (100 g): | VO | 203 | ml |
| | V10 | 190 | ml |
| | V500 | 184 | ml |
| | V1250 | 182 | ml |
| Packing ability | V10-500 | 6 | ml |
| Apparent density (g/ml) | m/VO | 0.493 | |
| | m/V1250 | 0.550 | |
| Residual humidity (%) | | 1.18 | |
| (3 g -15 min - 100° C.) | | | |

b) Size Distribution of the Grain on Superposed Sieves

The size distribution of the grain is shown in Table 5 below.

TABLE 5

| Mesh size | Rejects (%) | Cumulative rejects (%) |
|---|---|---|
| (μm) | 23419 | 23419 |
| 710 | 8.90 | 8.90 |
| 500 | 7.70 | 16.60 |
| 355 | 8.30 | 24.90 |
| 250 | 14.00 | 38.90 |
| 180 | 21.10 | 60.00 |
| 125 | 26.10 | 86.10 |
| 90 | 10.30 | 96.40 |
| bottom | 3.60 | 100.00 |

c) Pharmacotechnical Features of the Final Mix Before Compacting.

The pharmacotechnical features of the final mix are shown in table 6 below,

TABLE 6

| Test | | Batch |
|---|---|---|
| Flow (100 g) | | 4.38 |
| Bulk volume (100 g) | V0 | 195 |
| | V10 | 177 |
| | V500 | 171 |
| | V1250 | 169 |
| Packing ability (ml) | V10-500 | 6 |
| Apparent density (g/ml) | m/V0 | 0.51 |
| | m/V1250 | 0.59 |
| Residual humidity (%) | | 2.28 |
| (3 g - 15 min - 100° C.) | | |

d) Size Distribution of the Grain on Superposed Sieves.

The size distribution of the grain is shown in Table 7 below.

TABLE 7

| Mesh size (μm) | Rejects (%) | Cumulative rejects (%) |
|---|---|---|
| | 23419 | 23419 |
| 710 | 6.96 | 6.96 |
| 500 | 7.16 | 14.12 |
| 355 | 7.26 | 21.37 |
| 250 | 12.92 | 34.29 |
| 180 | 21.77 | 56.06 |
| 125 | 25.15 | 81.21 |
| 90 | 10.44 | 91.65 |
| Bottom | 8.35 | 100.00 |

The tablets obtained exhibits the features shown in table 8 below.

TABLE 8

| | Standard | | |
|---|---|---|---|
| | Metformin | Metformin | |
| Check | 500 mg | 1000 mg | Reference |
| Features | | | |
| Aspect | Round white tablet | Round white tablet | — |
| Color | | | — |
| Tests | | | |
| Residual Humidity* | <5.0% | <5.0% | |
| Average mass (mg) | 617.50 to 682.50 | 1235.00 to 1365.00 | Eur. Ph (2.9.5) |
| Mass uniformity | complies | complies | Eur. Ph (2.9.5) |
| Subdivision | — | complies | Eur. Ph (2.9.5) |
| Break up 37° C. | ≦3 minutes | ≦3 minutes | Eur. Ph. |
| | | | Monograph(0478) |
| Break up 20° C. | ≦3 minutes | ≦3 minutes | Eur. Ph.Monograph |
| | | | (0478) |
| Dispersion fineness | <710 μm | <710 μm | Eur. Ph. Monograph |
| | | | (0478) |
| in vitro dissolution | ≧75% at 15 min | ≧75% at 15 min. | Eur.Ph.(2.9.8) |
| a buffer pH 6.8 | | | |
| Resistance to | 40 to 80 N | 100 to 1410 N | Eur.Ph.(2.9.7) |
| crushing | | | Eur.Ph.(2.9.6) |
| Friability | 1.0% | 1.0% | |
| Uniformity of content | complies | complies | |
| Identification and | | | |
| dosage (HPLC) | | | |
| Average mass of | Between 475.0 | Between 950.0 and | |
| active substance | and 524.0 mg | 1050.0 mg | |

### Example 12

### In Vitro Release Profile of the Tablets from Batch 23421

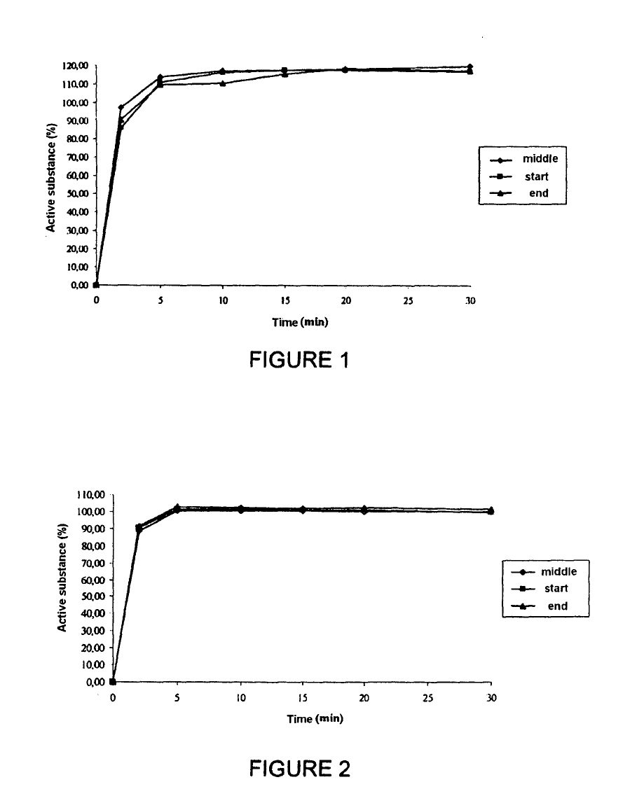

The release profile of tablets from batch 23421 has been measured versus time. The values of the measures have been gathered in table 9 below and illustrated by FIG. 1. The tablets have been analysed at the start middle and end of compression step.

TABLE 9

| | Active substance (%) | | |
|---|---|---|---|
| Time (min) | Middle | Start | End |
| 0 | 0.00 | 0.00 | 0.00 |
| 2 | 97.01 | 85.73 | 90.35 |
| 5 | 113.92 | 110.91 | 109.67 |
| 10 | 116.96 | 116.11 | 110.59 |
| 15 | 117.54 | 117.37 | 115.61 |
| 20 | 117.94 | 117.94 | 118.33 |
| 30 | 119.47 | 116.48 | 116.90 |

### Example 13

### In Vitro Release Profile of the Tablets from Batch 23422

The release profile of tablets from batch 23422 has been measured versus time. The values of the measures have been gathered in table 10 below and illustrated by FIG. 2. The tablets have been analysed at the start middle and ression step.

TABLE 10

| | Active substance (%) | | |
|---|---|---|---|
| Time (min) | Middle | Start | End |
| 0 | 0.00 | 0.00 | 0.00 |
| 2 | 88.28 | 90.51 | 91.14 |
| 5 | 100.56 | 101.54 | 102.85 |
| 10 | 100.67 | 101.21 | 102.43 |
| 15 | 100.38 | 100.96 | 102.05 |
| 20 | 100.12 | 100.97 | 102.18 |
| 30 | 99.99 | 99.33 | 101.35 |

### Example 14

### Comparative Analysis of in Vivo Pharmacokinetics Between Metformine Tablets Dosed at 500 mg According to the Invention and Metformine Tablets Marketed Under the Trade Name Glucophaqe® at the Same Dosages

A group of 26 individuals, between 18 and 55 years of age, were selected for the study and were divided into two subgroups (Crossover randomised study) respectively a first group treated with orodispersible or dispersible mefformine tablets dosed at 500 mg according to the invention and a second subgroup treated with two film-coated metformine tablets marketed under the trade mark Glucophage®.

10 ml samples of venous blood were collected from each individual, before the oral ingestion of the tablets and at the times 0.5; 1; 1.5; 2; 2.33; 2.67; 3; 3.33; 3.67; 4; 4.5; 5; 6; 8; 10; 12; 16; 24 and 36 hours after ingestion of the fenofibrate tablet.

The concentration of metformine, expressed in ng/ml, was measured in each blood sample taken.

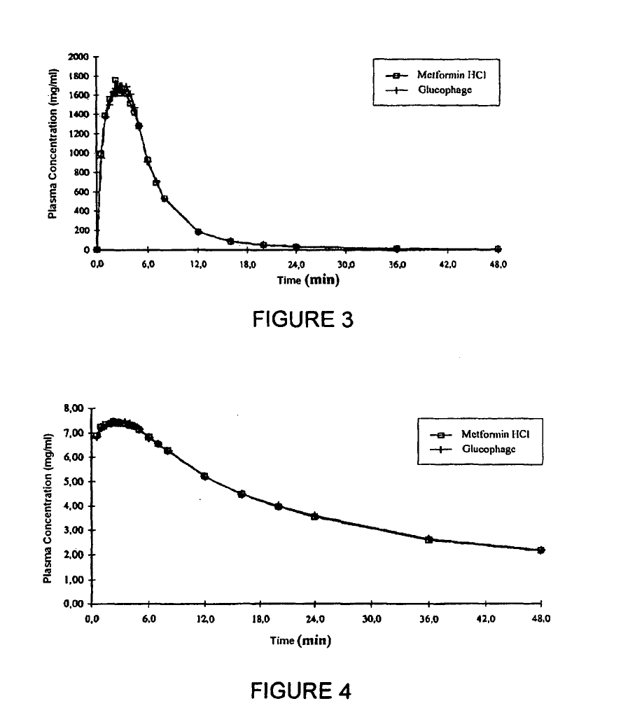

The mean concentration of metformine for all of the individuals was also calculated after the treatment by metformine tablets dosed at 500 mg (administered in two takes of 500 mg) according to the invention and after the treatment by film-coated metformine tablets dosed at 500 mg (administered in two takes of 500 mg) marketed under the trade mark Glucophage®. The variation curve of plasma fenofibric acid arithmetic mean concentration versus time was plotted, and presented in FIG. 3.

The variation graph of the napierian logarithm (Ln) of metformine plasma concentration versus time is shown in FIG. 4.

The maximum plasma concentration (Cₘₐₓ), the time until the maximum plasma concentration has been reached (Tₘₐₓ) and the area under the plasma concentration curve (AUC) have been measured for each treatment.

The values for these variables, means and ranges thereof were calculated for each treatment step and presented in following Table 11 below.

TABLE 11

| | Orodispersible or | | | |
|---|---|---|---|---|
| | dispersible | | Conventional tablets | |
| | tablets | | Reference | |
| | Test (Metformine HCl) | | (Glucophage ®) | |
| Parameters | Mean ± SD | Cl % | Mean ± SD | Cl (%) |
| AUC₀₋₄ (ng-h/mL) | 12469.38 ± 3978.20 | 31.90 | 12557.16 ± 4060.17 | 32.33 |
| AUC_{0-inf} (ng-h/mL) | 12698.98 ± 4036.25 | 31.78 | 12729.53 ± 4055.31 | 31.86 |
| Cₘₐₓ (ng/h/mL) | 1974.83 ± 887.24 | 44.93 | 1929.79 ± 603.57 | 31.28 |
| Residual area (%) | 1.80 ± 2.04 | 113.08 | 1.58 ± 1.58 | 105.39 |
| Tₘₐₓ (h) | 2.61 ± 0.91 | 34.74 | 2.39 ± 0.94 | 39.30 |
| Tₘₐₓ* (h) | 2.50 ± 1.38 | — | 2.50 ± 0.94 | — |
| Kₑₗ (h⁻¹) | 0.0777 ± 0.0385 | 49.52 | 0.0741 ± 0.0320 | 43.17 |
| T_{1/2 ck} (h) | 11.84 ± 7.16 | 60.44 | 11.20 ± 5.00 | 44.66 |

*interquartile median and means are depicted
AUC₀₋ₜ: area under the plasma concentration curve measured between 0 and 24 hours
AUC_{0-inf}: area under the plasma concentration curve calculated by extrapolation to the infinite.

The variables of table 11 have been compared in table 12 below.

TABLE 12

| | AUC₀₄ | AUC₀₋ | Cₘₐₓ |
|---|---|---|---|
| Ratio* | 74.6-88.2 | 72.5-85.6 | NS* |
| I.C. geom 90%* | NS | NS | NS |
| I.C. intra | 86.3-97.1 | 80.7-90.8 | NS |

*IC geom.: geometric confidence interval at 90% calculated on napierian logarithmic-transformed values
*Ratio: calculated with least squares method according to the following formula: e^{(Metformin HCl-Glucophage ®)} × 100

The combined results of table 12 above, show that the in vitro pharmacokinetic profile of metformine, for individuals treated with mefformine tablets dosed at 500 mg (administered in two takes of 500 mg) according to the invention is identical to the pharmacokinetic profile of individuals treated by film-coated metformin tablets marketed under the trade mark Glucophage ®.

1. A dispersible and orodispersible solid pharmaceutical composition having the form of particles with a size lower than 710 μm, containing the mefformin active ingredient, characterized in that it comprises:
a) from 65% to 90% in weight of the mefformin active ingredient, optionally under the form of a salt, or a combination of the mefformin active ingredient with a hypoglycemic active ingredient;
b) from 0.5 to 4% in weight of a binding agent or a combination of binding agents;
c) from 1% to 12% in weight of a disintegrating agent or a combination of disintegrating agents;
d) from 0% to 10% in weight of a diluting agent or a combination of diluting agents;
e) from 0.05% to 3% in weight of a sweetening agent or a combination of sweetening agents; and
f one or more additional excipients,
the weight percentages being expressed based on the total weight of said composition.

2. A composition according to claim 1, characterized in that it so comprises from 0.01% to 6% in weight of a flavouring agent, or a combination of avouring agents.

3. A composition according to claim 1, characterized in that the binding agent(s) is (are) selected amongst polyvinylpyrrolidone, sodium carboxymethylcellulose, alginic acid, hydroxypropylmethylcellulose and polyethylene oxide.

4. A composition according to claim 1, characterized in that the disintegrating agent(s) is (are) selected amongst sodium croscarmellose, cross-linked polyvinylpyrrolidone, sodium starch glycolate, wheat or corn starch and pre-gelatinized starch.

5. A composition according to claim 1, characterized in that the diluting agent(s) is (are) selected amongst lactose, mannitol, cellulose, microcrystalline cellulose and calcium carbonate.

6. A composition according to claim 1, characterized in that the sweetening agent(s) is (are) selected amongst gluconate, aspartame, cyclamate, sodium saccharinate, xylitol and maltitol.

7. A composition according to claim 2, characterized in that the flavouring agent(s) is (are) selected amongst fruit flavour, mint flavour, anise flavour, honey flavour, vanilla flavour, tea flavour, and verbena flavour.

8. A composition according to claim 1, characterized in that the mefformin active ingredient has the form of a salt selected amongst the phosphate, sulfate, hydrochloride, salicylate, maleate, benzoate, ethanedisulfonate, fumarate, succinate, chlorophenoxyacetate, embonate and glycolate salts.

9. A composition according to claim 1, characterized in that the hypoglycemic active ingredient, when present, is selected amongst glicazide, glipizide, chlorpropamid, glimepiride, glibenclamide, and derivatives thereof.

10. A composition according to claim 1 characterized in that it comprises, additionally, a PPAR Gamma agonist (peroxisome proliferator-activated receptor gamma) or Glitazone, selected amongst rosiglitazone, pioglitazone, and balaglitazone and derivatives thereof.

11. A composition according to claim 1 characterized in that it comprises, additionally, a PPAR Gamma and Alpha agonist or Glitazar selected amongst terapglitazar, muraglitazar, and ragaglitazar and derivatives thereof.

12. A composition according to claim 1 characterized in that it comprises, additionally, a hypocholesterol agent of fibrate type, such as fenobibrate and derivatives thereof.

13. A composition according to claim 1 characterized in that it comprises, additionally, an active ingredient selected amongst a dipeptidyl peptidase inhibitor (DPPIV).

14. A composition according to claim 1 characterized in that it comprises, additionally, acarbose or derivative thereof.

15. A composition according to claim 1, characterized in that it comprises:
a) from 65% to 80% in weight of the mefformin active ingredient, optionally under the form of a salt, or a combination of the metformin active ingredient with a hypoglycemic active ingredient;
b) from 0.5 to 4% in weight of a water-soluble polyvinylpyrrolidone with a molecular ranging from 44,000 to 54,000;
c) from 1% to 10% in weight of a water-insoluble cross-linked polyvinylpyrrolidone;
d) from 0.5% to 10% in weight of a diluting agent or a combination of diluting agents;
e) from 0.05% to 3% in weight of a sweetening agent or a combination of sweetening agents; and
f) one or more additional excipients,
the weight percentages being expressed based on the total weight of said composition.

16. A composition according to claim 1, characterized in that it comprises (i) an internal core comprising the active ingredient or the combination of active ingredients, in association with one or more excipients and (ii) an external layer comprising the sweetening agent.

17. A composition according to claim 16, characterized in that the internal core accounts for 75% to 85% in weight and in that the external layer accounts for 15% to 25% in weight, based on the total weight of the composition.

18. A composition according to any of the claims 16 or 17, wherein:
(i) the internal core comprising comprising:
a) from 65% to 80% in weight of the mefformin active ingredient, optionally under the form of a salt or a combination of the mefformin active ingredient with a hypoglycemic active ingredient, and
b) from 0.5% to 4% in weight of a binding agent or a combination of binding agents; and
(ii) the external layer is non-film coated and comprises comprising:
a) from 0% to 10% in weight of a diluting agent or a combination of diluting agents;
b) from 1% to 10% in weight of a disintegrating agent or a combination of disintegrating agents; and
c) from 0.05% to 3% in weight of a sweetening agent or a combination of sweetening agents;
the weight percentages being expressed based on the total weight of said composition.

19. A composition according to claim 18, characterized in that the binding agent is a water-soluble polyvinylpyrrolidone with a molecular weight ranging from 44,000 to 54,000.

20. A composition according to claim 18, characterized in that the disintegrating agent is a water-insoluble cross-linked polyvinylpyrrolidone.

21. A composition according to claim 18, wherein:
(i) the internal core comprises:
a) from 76% to 77% in weight of the mefformin active ingredient, optionally under the form of a salt or a combination of the mefformin active ingredient with a hypoglycemic active ingredient, and
b) from 2.5% to 3.5% in weight of a water-soluble polyvinylpyrrolidone with a molecular weight ranging from 44,000 to 54,000; and
(ii) the external non film-coated layer comprises:
a) from 6.5% to 7.5% in weight of a diluting agent or a combination of diluting agents;
b) from 4.5% to 5.5% in weight of a water-insoluble cross-linked polyvinylpyrrolidone; and
c) from 0.5% to 2.5% in weight of a sweetening agent or a combination of sweetening agents;
the weight percentages being expressed based on the total weight of said composition.

22. A composition according to claim 1, characterized in that it consists in:
(i) an internal core comprising:
a) 76,92% in weight of the metformin hydrochloride active ingredient, and
b) 3.08% in weight of a water-soluble polyvinylpyrrolidone with a molecular weight ranging from 44,000 to 54,000; and
(ii) an external non film-coated layer comprising:
a) 7% in weight of a diluting agent or of a combination of diluting agents;
b) 5% in weight of a water-insoluble cross-linked polyvinylpyrrolidone;
c) 2% in weight of a sweetening agent or a combination of sweetening agents;
d) 5% in weight of a flavouring agent or a combination of flavouring agents; and
e) 1% in weight of a preservative;
the weight percentages being expressed based on the total weight of said composition.

23. A hydrodispersible non film-coated pharmaceutical tablet, characterized in that it consists in a composition according to claim 1.

24. A tablet according to claim 23, which pharmacokinetic profile established from two tablets, each dosed at 500 mg, is characterized by an area under the plasma concentration curve measured in vivo (AUC) ranging from 10000 ng.h/ml to 16250 ng.h/ml and preferably of about 12500 ng.h/ml

25. A tablet according to claim 23 or 24, which pharmacokinetic profile established from two tablets, each dosed at 500 mg, is characterized by a maximum plasma concentration value (Cₘₐₓ) ranging from 1600 ng/ml to 2600 ng/ml and preferably of about 2000 ng/ml.

26. A tablet according to any one of claims 23 or 24, which pharmacokinetic profile established from two tablets, each dosed at 500 mg, is characterized by a Tₘₐₓ value ranging from 2 h and 3.25 h and preferably of about 2.5 h

27. A tablet according to claim 23, dosed at 500 mg in mefformine chlorhydrate, giving results that can be extrapolated to the compositions of the same type and comprising lower doses in mefformine chlorhydrate, metformine chlorhydrate absorption in human beings, being linear from 0 to 1000 mg.

28. A tablet according to claim 23, dosed at 500 mg, releasing between 50% and 100% of the active ingredient dose and preferably at least 80% of the metformine chlorhydrate dose in 5 minutes in a physiological buffer medium pH 6,8 at 37° C.

29. A method for preparing a hydrodispersible non film-coated pharmaceutical tablet characterized in that it comprises the following steps of:
a) preparing (i) an internal core comprising a dispersible and orodispersible solid pharmaceutical composition having the form of particles with a size lower than 710 μm, containing the mefformin active ingredient, the composition comprising:
1) from 65% to 90% in weight of the mefformin active ingredient, optionally under the form of a salt, or a combination of the mefformin active ingredient with a hypoglycemic active ingredient;
2) from 0.5 to 4% in weight of a binding agent or a combination of binding agents;
3) from 1% to 12% in weight of a disintegrating agent or a combination of disintegrating agents;
4) from 0% to 10% in weight of a diluting agent or a combination of diluting agents; and
5) one or more additional excipients,
through wet granulation of a mixture of metformin appropriate amounts, optionally under the form of a salt or a combination of the mefformin active ingredient with a hypoglycemic active ingredient, and a binding agent;
b) drying the granules obtained in step a);
c) adding to the granules obtained in step b) the mixture of excipients forming (ii) an external layer comprising from 0.05% to 3% in weight of a sweetening agent or a combination of sweetening agents; the weight percentages being expressed based on the total weight of said composition; and
d) performing a compression of the granules obtained in step c).

30. A method for preparing a hydrodispersible non film-coated pharmaceutical tablet characterized in that it comprises the following steps of:
a) preparing (i) an internal core comprising a dispersible and orodispersible solid pharmaceutical composition having the form of particles with a size lower than 710 μm, containing the mefformin active ingredient, the composition comprising:
1) from 65% to 90% in weight of the mefformin active ingredient, optionally under the form of a salt, or a combination of the metformin active ingredient with a hypoglycemic active ingredient;
2) from 0.5 to 4% in weight of a binding agent or a combination of binding agents;
3) from 1% to 12% in weight of a disintegrating agent or a combination of disintegrating agents;
4) from 0% to 10% in weight of a diluting agent or a combination of diluting agents; and
5) one or more additional excipients,
through dry granulation of a mixture of metformin appropriate amounts, optionally under the form of a salt or a combination of the metformin active ingredient with a hypoglycemic active ingredient, and a binding agent;
b) compacting the dry granules obtained in step a);
c) adding to the granules obtained in step b) the mixture of excipients forming (ii) an external layer comprising from 0.05% to 3% in weight of a sweetening agent or a combination of sweetening agents; the weight percentages being expressed based on the total weight of said composition; and
d) performing a compression of the granules obtained in step c).

31. A method for preparing a hydrodispersible non film-coated pharmaceutical tablet characterized in that it comprises the following steps of:
a) preparing a mixture of (i) an internal core comprising a dispersible and orodispersible solid pharmaceutical composition having the form of particles with a size lower than 710 μm, containing the mefformin active ingredient, the composition comprising:
1) from 65% to 90% in weight of the metformin active ingredient, optionally under the form of a salt, or a combination of the mefformin active ingredient with a hypoglycemic active ingredient;
2) from 0.5 to 4% in weight of a binding agent or a combination of binding agents;
3) from 1% to 12% in weight of a disintegrating agent or a combination of disintegrating agents;
4) from 0% to 10% in weight of a diluting agent or a combination of diluting agents; and
5) one or more additional excipients,
through dry granulation of a mixture of the metformin appropriate amounts, optionally under the form of a salt or a combination of the mefformin active ingredient with a hypoglycaemic active ingredient, and the binding agent;
b) adding to the granules obtained in step a) the excipient mixture forming (ii) an external layer comprising from 0.05% to 3% in weight of a sweetening agent or a combination of sweetening agents; the weight percentages being expressed based on the total weight of said composition; and
c) performing a compression of the granules obtained in step b).
